(19) United States
(12) Patent Application Publication
(10) Pub. No.: US 2016/0338882 A1
(43) Pub. Date: Nov. 24, 2016

(54) Circular Gusset in Reusable Cloth Diapers

(71) Applicant: Sarah Downey, Barrie (CA)

(72) Inventor: Sarah Downey, Barrie (CA)

(21) Appl. No.: 14/686,841

(22) Filed: Apr. 15, 2015

(51) Int. Cl. A61F 13/494; A61F 13/49

(52) U.S. Cl. CPC A61F 13/49473; A61F 13/49004; A61F 2013/49074; A61F 2013/4948

(57) ABSTRACT

A reusable cloth diaper including a continuous perimeter elasticized gusset located within the interior cloth of the diaper against the wearer's body.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field of infant diapers and more particularly in the field of infant reusable cloth diapers.

Reusable cloth diapers are a commonly-used washable item that is meant to contain and absorb a baby's liquid and solid waste. Reusable cloth diapers can be made of a variety of materials but generally have at least two components including an absorbent part as well as a waterproof part.

Formerly, reusable cloth diapers were comprised of a simple piece of cloth that was folded and pinned into place on the baby, sometimes covered by a set of waterproof pants. In recent years, reusable cloth diapers have come to more closely resemble the shape and features of disposable diapers. These reusable cloth diapers usually have a permeable layer attached to a waterproof layer with elastic sewn along the back and through the leg with an absorbent material in between the two layers in an hourglass type shape that conforms to the baby's body and is secured with hook and loop or snap closures.

The cloth diaper industry, has employed different sewing techniques with elastic commonly called “gussets” or “inner gussets,” as a method of better containing the baby's bodily fluids within the perimeter of the diaper. Typically, inner gussets are a pair of elastics sewn on both sides of the lining of the diaper that create a barrier along the outer leg elastic improving the performance. Nonetheless, leaks continue to be a problem for cloth diaper users as fluid and waste can find their way around the ends of the inner gussets and therefore there is still a great need to enhance the ability of cloth diapers to contain leaks. Prior art includes patent number U.S. Pat. No. 8,425,483 B2 issued on Apr. 23, 2013 to Julie Ekstrom.

### SUMMARY OF THE INVENTION

The present invention of the circular gusset is an elastic sewn into a circular shape and then sewn into the inner permeable lining fabric of the cloth diaper in an oval shape, creating an all-around elastic barrier on the inside of the diaper. The objective of the present invention is to trap all of the baby's human waste by containing it within the circular walls of the gusset thus preventing leaks from escaping the diaper in all directions.

### BRIEF DESCRIPTION OF THE DRAWINGS

FIG. 1 is a perspective view of a reusable cloth diaper according to the described embodiment;

FIG. 2 is a top plan view of the reusable cloth diaper illustrating the interior of the diaper;

FIG. 3 is a bottom plan view of the circular gusset reusable cloth diaper illustrating the exterior of the diaper;

FIG. 4 is a cross-section of the described diaper;

FIG. 5 is a top plan view of a pattern detail of an interior panel as viewed from the rough side;

FIG. 6 is a view of the sewn elastic elongate

### DETAILED DESCRIPTION OF THE INVENTION

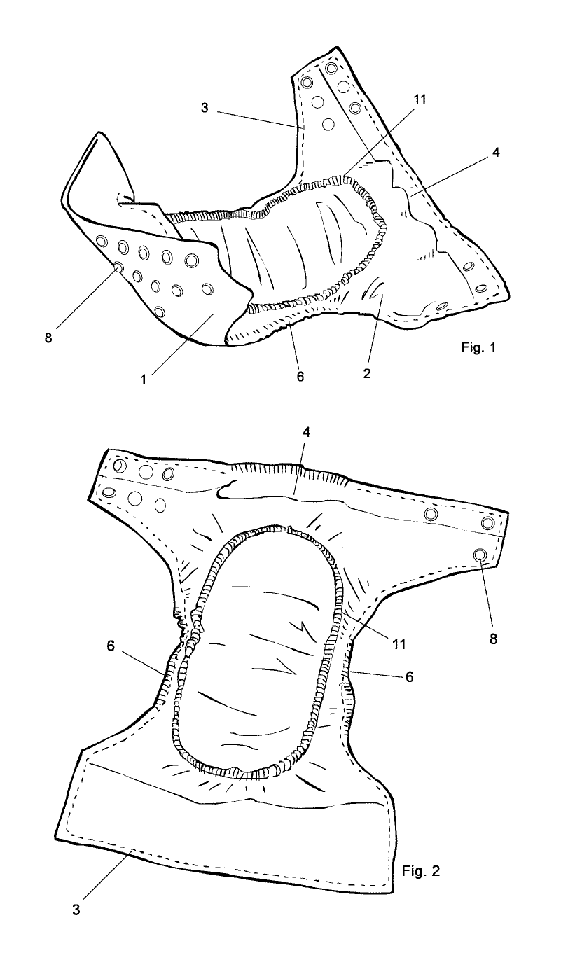

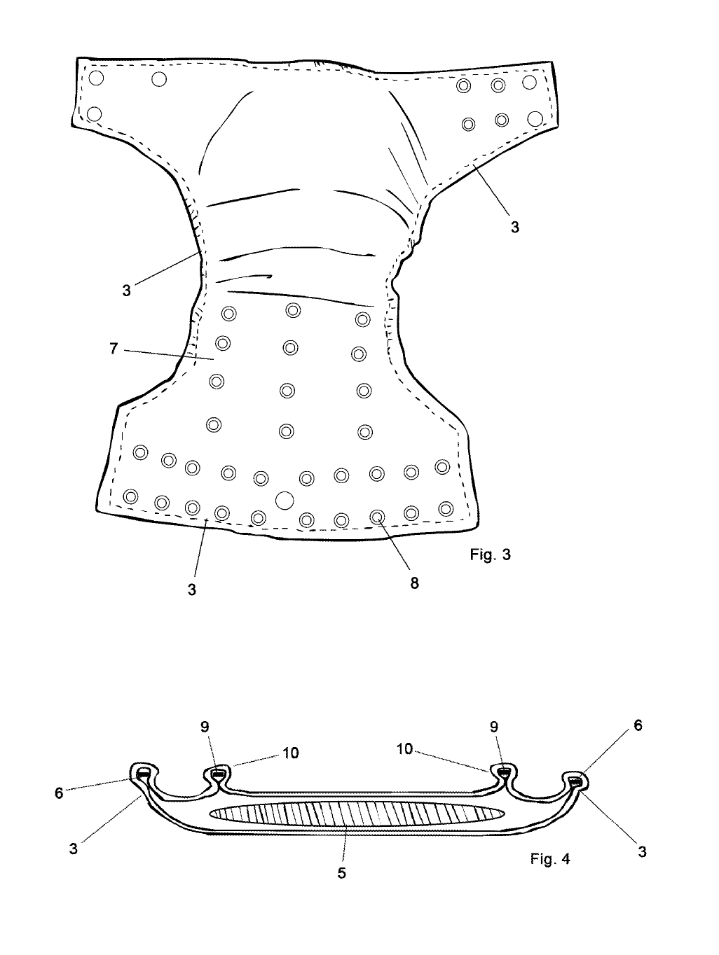

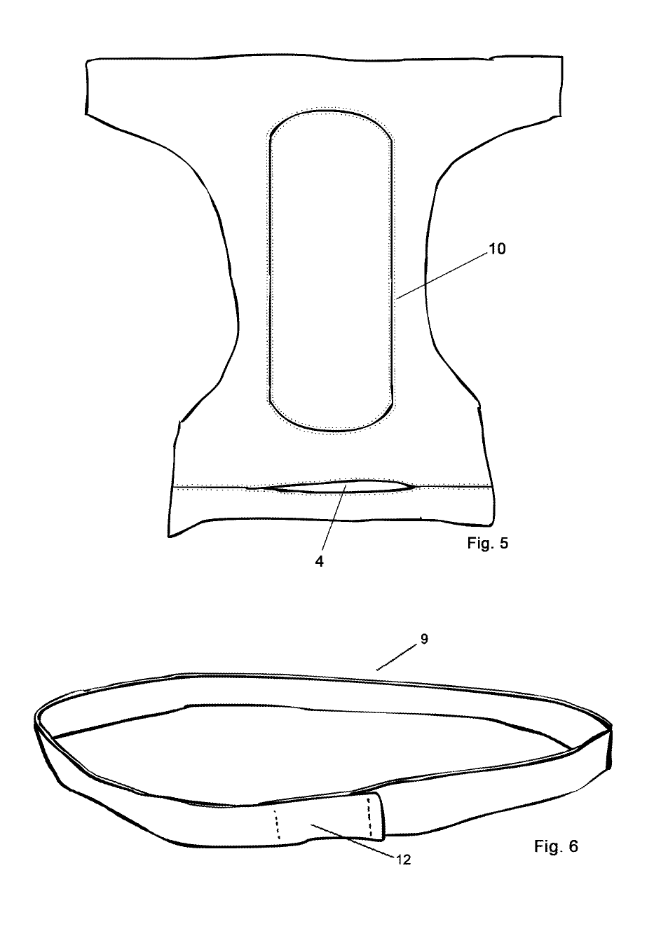

One embodiment of the circular gusset reusable cloth diaper includes an outer waterproof material FIG. 1.1 that is sewn to an inner permeable FIG. 1.2 material around the edges of both materials FIG. 1.3, 2.3, 3.3, 4.3 with at least one opening FIG. 1.4, 2.4, 5.4 allowing for insertion of a removable third material that is absorbent FIG. 4.5. Elastic is sewn along some of the edges of the waterproof and permeable material FIG. 1.6, 2.6, 4.6 to allow the diaper to conform to the baby's body. This diaper is made with snaps to adjust the size FIG. 3.7 and to attach it to the baby FIG. 1.8, 2.8. This diaper is meant to be worn by infants to contain their bodily fluids and can then be washed and reused repeatedly. The continuous perimeter gusset is created by sewing the ends of an elongate elastic together FIG. 6.12 to create an elasticized circle FIG. 6.9, 4.9 that is then placed in a gathered channel FIG. 4.10, 5.10 that is created on the top side of the permeable lining material close to the edges all around the inside of the diaper FIG. 1.11, 2.11. The result is an oval shaped elasticized perimeter with elevated edges FIG. 1.11, 2.11 that traps bodily fluids and waste in the center of the diaper.

1. A reusable cloth diaper including:
an inner cloth layer against the skin intended to wick away moisture; and
an outer cloth layer intended to act as a water barrier; and
an additional cloth layer positioned in between said inner cloth layer and outer cloth layer intended to absorb moisture; and
a continuous elasticized perimeter formed within said inner cloth layer comprised of a elongate elastic sewn into a closed circle and placed within a continuous channel created in said inner cloth layer

2. A reusable cloth diaper including:
a cloth material intended to absorb moisture; and
a continuous elasticized perimeter formed within said cloth material comprised of a elongate elastic sewn into a closed circle and placed within a continuous channel created in said cloth material
